SEQUENCE LISTING (67 sequences)

SEQ ID NO: 1 - RNA, 1641 nt, Tilapia lake virus
  source 1..1641: mol_type = viral cRNA
gcaaatatttctctcattcgcctatatagttacttagcacccagcggtgggctggactgctttataaatagcatagttaaagtttgtgggttgttccaagcctaaggacttcaagtgagcctgcctagctgctctgatcttgctaacgtctgactctgatacgaggagcctatgagggaccagggggtccaagtctgagagagcctccagtcgggttctccttctgcgagtctttgaagcaaggacagacaggactttctgatctatagtgtctgggccatcggggtgcacctggtaccaaaatgggtggcaatcccaagtcagtagagattgaaagctaattacagtcttccctcccaggcatgtggggaggaagtcctcattcctcgttgtgtaagtggggttgttatacctataggcacattcctgctgctttatctggaacagacgttctgctgagtggaatggcataactcctgagtcaacagcgccagataacatagctgcagtctgtttcagagtcacaagtggtcccctgaaagggacaccagcagtacaacccaaccctgtggctaggtcaccgtccctagtgagggtacacgagtttatttccagtgaggcaactgaaatgtaactcttctttagcgacaagttatgacagcttgcgaaaagcaggtcctctatctcgcgtaattcagcaggcgagttaaacgatgttataaagtcgtcgctgaaagacaggaatctgtcagtcgtcccctgcaatgccaaggtggcagttgcgttaaacattcccatcaacatacctcctggacactctactagagtgtctttaatgagaacgcatttcctgcgcaccatgtaagcgagcatgactccgattgaccccatgcctattgcatcccacacttttagcagtgtgtctgcgtcgattgcctcattatacttagtacagtcgccattaagaacatgttttcttgactctacaacctgtgcggccatcatagcccgttgttctgcactagcagtagcggtgtacactgctgagacctttgacagcacagatctagcgtgcgtctctactacagaggaaagcaatctgaagaatggatgtgacagaaagatagctctgctcttgggtttacccctctcctggtcgcgaagtgcgcacttaaagtacctgccatgagaaagtaactcctcgacgctcgtgtcacctatcttttcagatagggattctactgtcaaaaccttacatatgagctcagatggagaactacaccttgttgtgttcaacaggcccgtcataccgcgggtgagcgtggtacttttgcatacaactctgccctccttctctataacccaacagcaactcctttctgaataaaggtcggaatcgactatcctcagtggactttctccctgattaggcagcttgcttaagtccccagtgtggacagcattgtacgattttcctgtcatgatttcagacgctctgtctaatgactccctcgctgctgaatccggtgccttcattgaggtcgggcctgataacaggttacctttgcaaactccttcttgaaatgcccacataccttatggtagtacagtaatagtgcgtaattaagagataacgtttgg

SEQ ID NO: 2 - RNA, 1471 nt, Tilapia lake virus
  source 1..1471: mol_type = viral cRNA
gcaaatctttctcattattaccatatatatagtgaaggcttttgccaagaaaccctactgatttagatccatattttccgctacctcccggaaattctcaaccctcatatctttaaagcgatcctcctgcactgccacttttcgcttctttgcgtacgtgcctggactacatgagatgagaagcattgccttcctctttctacagtaaaatgtaccgttcggaggtttctgaggaaggtcctcgtaacccatccactttgatctcgagagaacagtgaccttcgccttgtcaaggtagaaccctctaaccacatcgtccaggtttacaaccaggccctctatcgtaatgtacctctcgggagaacgtaatgcctttatattgagcttcctcttgtcgagatcgcattcaaagttttcacctacaggtgtgatgagatttgcaagcccttgcagtatagatctagaggcgggcgtccccctactgtacgctgctctgagcctgtccagggcggtatgtattgagcctgagacatttgcactaccttgcggtccaaggttcctcactgaaagcgcttgttgaaaaagtgtttcctgtgatatgtggttagctgttgcataccctacgtccctgtggtacacccaaccctggtcgcagaaggccctgcctccaaatggatctagtgcagtaggtgccctagcaaaactagcttcccacactttcctacaagggcactcagctgctgctatagatggatcctcttttgggaccagattcagcctctttgcaagacgttttgccaaccaaaacgaaggagacttacttgctaggaacttagcatcctcgacagcgacgaagtgttggaacccgtcatacatgtgccagaattcgccgatttttatcacaaatccaagtgaatctttgaaagaacctggcccacaacatatgaacccgcatggagtggtaggaattgctctcacgaagccggattcgcatgtatcaaatgctctatcagccaaagtgtaccctagagccgctaagcccctgtgaaatttgtcattatctgcactcgagtaagctacaggctcattgactaccctgtcttgcaaggcacctgagacgctgtcaccatcatagcccttgttgtaaagatcaattgcttcctcctgagtcaaaccgggtctgaacgacagctgactcctcagcttagactttttcacagcaaccctggactctaattttggccgaatgggcaaaagaggcaaatctttatccaagaactggtttagggcgttcctaaaacagaatgacttccttagagacttgtcagccgtaagtaagcttctgtgcacatctttgacagtatgagagcgatattcggttattgtgacctcagttctgcccttgaatgatttcccaaactgactcatttttcagaagtaaatgtatttggtaatagagagtaaaatttgg

SEQ ID NO: 3 - RNA, 1371 nt, Tilapia lake virus
  source 1..1371: mol_type = viral cRNA
gcaaatttttcccataatcctctattagaacgtcgtaacctttagcgaaggcgtcgaaagcgatcatctcgcaaatgggtgtactgtcatccgcaatcttactgcacaaagtgaataataaagtgagcttaagggtattgtaccccttatctcagaagccagctggtagcctttcctgaactcgtccttacagacgctaagtgctagccggtgcctattaaaatgttgagccactgcttggctaacccttgtagagtcgaggcattccagaagtaagatgacgtcccatcttgtctcaagaccactagctctgtccagatcacccttcctacttatgggaggtagttccaacatatccagcttgtaaatttctcgggtactcacaaagtcttgctcccctatattagccgattgcttgggatctaggtgcatcacatgcacagctgccctgtacccgtcaaactcaaaatcgtgttcacarccaggtttacttagacctacaactaagtggtgagtggaggcggttggtctccttttactgtgctttccagagtcgcgcatgaccggtacagctagtatgctggtattatcgctatgcagtactttccctgcctgagttgtgcttctagcaatcaacatcaaaagctcacgagcaagtggrgcactagccggtagaggtaatatcttctgtgtagcaggcttrtgagaagcaactgtatacctttgtatccaccctccattgcggaactcaaattctctatcacgtgcgtactcgttcagtataagttctcttgcctcttggtcaagaccacactcctcaccgcaggcgaggaactttgagcactcgaagaatccatattgcctctttagctcagctgtttccttggatatgtccgcaagtctgggtggtgccacccactcaatacgaggcttcgggccactctttggatgtggtagttccaatagccgttcccttagctcagcatcgtaggatgccttgtgcccaactttaacaaccccgtcgatctccaaataatccttgaattgtaccttacaaatctgctggtactcttgctcagagcaaacgtatgtagcatcattgttcaaaatgcaacgaactgttgcctttggaaatttccgcggctggccttccagctcaaaggcaatccacttattcttcaaacagtcataacagtcaccctctacggggactcctggacgtctctctactgtactgtaagaacttaggaaccttcggctcccttcgctataagtgaaatcgtcacagaaaaccccagttagctgtgcaaaccgcgagtccatttcaaaagaaagttaacggtctattaaggattaaggggtaatatttgg

SEQ ID NO: 4 - RNA, 1250 nt, Tilapia lake virus
  source 1..1250: mol_type = viral cRNA
gcaaatctttctccaattaccgtctaagaatttaccagttttagatgacttgttaaagaatatatagaaaacttgggctaagaggggcccttggcagcctgtgcagctttccgcgcatgggtggcctacttcgttgcctatctcccaacagcccctgctgtctccttgaggcagtgatacagactcttcccacttctcttacattttatgacaccgtcctgtttctgaatggcaatgcattcttccagcagattcgtgttccctgttccctcaatcctagagtcggcaattgctgagagcccgtggctagctaccaccaggaagcagcttcctgtagcttgcagaggtagaccaacaacaacaccaatactcccgtccccctgcgaggggctccaacactcagcaaggacaccgtcgcaaaggccccaagcaattgaggcaagcagctccctgagggtgacctggtcgcctatctgcagcctgtggccaatgatcagaaatctcggaggcgggattatagcagtctgattcaactttttgtagttctttcctctttctcctgctgctgctttctttgcaaagtagccaccaaccagccctgggcaagcacgataggaatccccactcagatatgggaaccattgggcgtcctccaaagtctgaatcctctcgtcgagagcaaccaaactgacgtacctagccttccagacctcaatgcgaacacacactcttaatagagagttatggtccttcatcatctgaatcatagctgcgtcccctgaggcttttatcaccttcatctttgagcccagtgcaggcacacaggttgcgcagtattcagtccatctgctggttaatataaggctccttccgaccctcatccctgcttctggtgccttccttgatttcttagcgttctccctctctctcttcttttcactggctctcagaaccttagcctcctgaccatcgagggagtgagtgtgcattttaaagagaaatgcagcagcatcagatatcacactgttgtcctccctgttaaaggaacttccaaacacatgaccacaagcttcgttgaaaacctcaaggtttcttgggagttcagcttgtacctgcgaagtgctgggtgaactttcatccattgctacctctggtgcaacagtgctggcagctgccatactagtctttgtagttctcaccattttcagtttattaagttagctattctgggtaataggagtaaactttgg

SEQ ID NO: 5 - RNA, 1099 nt, Tilapia lake virus
  source 1..1099: mol_type = viral cRNA
gcaaattttactctttttctcagtttaccactctatgacctaccaggaatagaagcttaaggatcaagataatggaagcagagggacttcgtcatcctcaagcgggcttttcttcttaagccgcttcttaatatagaaaaaggtcttgaaacacacaactcttagcctccggaatactaaaactttgatactcccatagaagggcacctgcttcaacagagaaacaagtttactgagcaatgaaagaaccatcagcgaaagcaccaggtaatagacaaacttgtatttctctagcatcccaaacactattgcaagaatagcttccccaaaggctgcaaattgctccttgaatttgcctatgttatcagatttacagacgagggatttaatcgagcaaaactctcctagcgtcataccggtgacttcccgtgtcaaagcttctaaatactcccaaatctcagaaacatctgcttgagctgcgtccgctaggccgatgaccccagcataattgtaaacacccatgccaattgctactgagctagcaagtaaacacagtataatgaggcctgctactacaaacgacttcggctgccttgaggccagcatggtcttgtagtcgtccacagtgggtctggtacagtcacactgacctggcagtacggtggaatacacctccaaatctctgcactcgctactgtggctgcacctggaaacgttgtataggacaaccccgctatcttcgtcaatcctaacttcagtagctctccaatcacctcttccccctgctttgggaagtttgtacccgagggctgggaagctgttaggtactgcatgtaacccagtgcagaacgacccacccgaggatggggttacaatgtagtgcacccccatagtgcaggagcaactttccatcctgaacttggatgcaaatccacgccttatttcactttctatctccctactttggtaggtattgtttaggtatgattgtaatcgctttatccttaaactttcatcacttgctagtgcgcaaactaggcagcttatcaggaccagcgcctgcatagctattggagtctgagataagagaaacatttgg

SEQ ID NO: 6 - RNA, 1044 nt, Tilapia lake virus
  source 1..1044: mol_type = viral cRNA
gcaaatatttctctcaatcaagcacttaaaactgtacctgggcatttcgcccacacgacaggacatatagtgtcacatgtatttattgattttacagcaggatcttactgttactggtggtgatatcgaaagatcaattacaggattcaatctacgcatttttgtgtagctgagaaaagtacgagctagaagataagcatttgacttcttataagcaacttcatcctgcatcgcggaccactcgctgatgagctggtgacgctttatttcggctggtagtaagacttccactgcggaaaagtggcacttgtatggggagtgattctcctccagtgactcaactaatatccaacactgttttgtactattgtctctgcagctccacttgtattcagttctgaagccgtttggcgtcatatatgttctatctatagtacgcttacagtttgtacaaagaccacgcgacattagcatacaggtgctaaagatgggcctcctcttcgaaaggcacagcgatgtctcaaacagtttagagcttatttttatagtacgtattttacctcggcccggtattccccggctctccaatatcttgtcggttacaggctccagttcttttctcgatattcccgggcacaaaacaaatcctccaagcataaagttcagcgtcggattacatcgtttctcgacaaagttgcagttcagatgatggagttccccttttgatccctccaaatcatacatgtagtatggagacccacctgagtctatgacttcgttgctaccatcaagtcccagaaccaccttctgtactgctggatccagataatcactactcgaatatgaaccttcaacagaatatttgtccccgtcaataactacgcttgacgtgtagtttgatctcccataaaaccgccacatattgtccacgcattgatcgcttccgctaaagagtgttgaaaacaaagtaagggttcttatcaccctaagccaactcattggataatcctgtagataaaaatgcatgcgagaggtaaaatttgg

SEQ ID NO: 7 - RNA, 777 nt, Tilapia lake virus
  source 1..777: mol_type = viral cRNA
gcaaatctttctctcatgctaccatccttagtgaacggtactgagtaccataagaagctctcagaccaattatccctgctttcaaaagtaattagttaacttagaaaggcctccccaataagacaacgaaacagctgggtaattagagttcaaacgtgattcctttagggattggcactaacccaactctgtggtaggaacaccacgattcattgacatcagttgcacccttacttatagcctctaggtatgtggcaaccctgctcaactcagtcagtggtcgagtgctggtcaccaacgctgatctcttggtccaatgtggaaactttggggatggcgcacagagccagtacttcgcttcccttctagtgtagaaggtagttgaacagagatgcatgtcccctttgactaaataggctcctttcgggcctcttactgctgtaccctcctgggcaagtacccttctgactggcaatccggggctcacttcatacttccatttcaaaggtaccttttctccaccatcggttgggtgactcgtcaatacaatccaggttgtgccatttgtattgggtgtaaacccagttgcacaaaagaaccatacgttcgaggcaccagagtaatgctctaggagcagtcgatggaacagcccagtctgacttccgctgtccttggggagggtgtttttgcgcgtgataattctctcaagctcaccaatcttgtaggacattgtgtctattactatacggtatgcaatgcaaagagagtaaaatttgg

SEQ ID NO: 8 - RNA, 657 nt, Tilapia lake virus
  source 1..657: mol_type = viral cRNA
gcaaatttttctcatcattacacaaatggagtagcttacctccctggggaaaatgacactgaagactcactttttatttaagcatttcacggaaatgattgatagcagcagaagtgtctgccttaagggccatcctgtcatcttgtgaaccttcagtgtccttcaaagcagttgccaggacctcctcgagaaaagcgagcgcagtgggtatgtcgacacagttaatcccagggagcttaagcttcatagcttgtttggcaatatccaggtactgcttccgattgaattcaaatctttgacccttgcggtttgttattataattgcgtcgactgtcacactgactcgcctcgagattcgtcccagcctttcctctatccgctttcgggtcatgccggcgtgcttaacaagcaggtgtttaaactttgtttcttctgagaggccttcgccaattgttagggaaagcaaatccggacctgcattgacaagaaactccagagcaactacagtgttgactgtgtctctagtcactgtcatgccgttgagcgtgaaatcgtagagcttcccttggccctctcttagtgttgggatttgagccatacttattcctggatatgctgtccaattggaaatgttagtgtagatgaggtaatatttgg

SEQ ID NO: 9 - RNA, 548 nt, Tilapia lake virus
  source 1..548: mol_type = viral cRNA
gcaaatctttctcacgtccttaaagtcatactttgccagagctgaagcttatccatttttactaaagctaaattattaatgtcacatggttcactacaatgttctagctgaggaatcagtaggttcgcggagcttcaattgcccctggaccgccctcataaagttctatcgccagccatgtcagatatcctccacatgacccttcttggtgcctgcctttccgatgttgaagtgtgcaaaattccgtgctccgttactacttggacccagctccctatcaccattgcactctcctgccaagttggctgactcagtgtcattaactctgagatctctgaacattctagacaatgttttcgatccaggatccagacttgaccaggttgtgatgtcttgccgccgcagcacaagctccaaggggggtaatggcacaaagtgacgtacgaccgactcatctctgtcaagattactgacacagaatctgtattttctatccatcgtgacatcaccaagcggaaaaagtaatcggacttgtgagtaaaatttgg

SEQ ID NO: 10 - RNA, 465 nt, Tilapia lake virus
  source 1..465: mol_type = viral cRNA
gcaaatctttccctctgacaccctgtatagttagcgttggcctgtggatacgaacgaaatcagaaccgatattaaggtgctaagactgcacgtcaagagacttctttccgaaatcttcggaaaatcgagataggtcactctgcccatcatcctctctgtcccttctgtttttgggattgaaatcaaccctagcccactctgggattgcagaatcacagtcgtccatctcgaggtcgacttcgtcaccccactctatattatcttcaccgctctcgtcagcaccatacctttcattcttccaacttcgcttctttgaagcagctttcttgcccttcttgatcttccgacttcttagtactaaacatcctgagctctcagcccccgagtcactgtcacttgacaaataatctgccacactcatcctggcttatagctatatttggtgttagtagggttaaaatttgg

SEQ ID NO: 11 - RNA, 1641 nt, Tilapia lake virus
  source 1..1641: mol_type = viral cRNA
ccaaacgttatctcttaattacgcactattactgtactaccataaggtatgtgggcatttcaagaaggagtttgcaaaggtaacctgttatcaggcccgacctcaatgaaggcaccggattcagcagcgagggagtcattagacagagcgtctgaaatcatgacaggaaaatcgtacaatgctgtccacactggggacttaagcaagctgcctaatcagggagaaagtccactgaggatagtcgattccgacctttattcagaaaggagttgctgttgggttatagagaaggagggcagagttgtatgcaaaagtaccacgctcacccgcggtatgacgggcctgttgaacacaacaaggtgtagttctccatctgagctcatatgtaaggttttgacagtagaatccctatctgaaaagataggtgacacgagcgtcgaggagttactttctcatggcaggtactttaagtgcgcacttcgcgaccaggagaggggtaaacccaagagcagagctatctttctgtcacatccattcttcagattgctttcctctgtagtagagacgcacgctagatctgtgctgtcaaaggtctcagcagtgtacaccgctactgctagtgcagaacaacgggctatgatggccgcacaggttgtagagtcaagaaaacatgttcttaatggcgactgtactaagtataatgaggcaatcgacgcagacacactgctaaaagtgtgggatgcaataggcatggggtcaatcggagtcatgctcgcttacatggtgcgcaggaaatgcgttctcattaaagacactctagtagagtgtccaggaggtatgttgatgggaatgtttaacgcaactgccaccttggcattgcaggggacgactgacagattcctgtctttcagcgacgactttataacatcgtttaactcgcctgctgaattacgcgagatagaggacctgcttttcgcaagctgtcataacttgtcgctaaagaagagttacatttcagttgcctcactggaaataaactcgtgtaccctcactagggacggtgacctagccacagggttgggttgtactgctggtgtccctttcaggggaccacttgtgactctgaaacagactgcagctatgttatctggcgctgttgactcaggagttatgccattccactcagcagaacgtctgttccagataaagcagcaggaatgtgcctataggtataacaaccccacttacacaacgaggaatgaggacttcctccccacatgcctgggagggaagactgtaattagctttcaatctctactgacttgggattgccacccattttggtaccaggtrcaccccgatggcccagacactatagatcagaaagtcctgtctgtccttgcttcaaagactcgcagaaggagaacccgactggaggctctctcagacttggaccccctggtccctcataggctcctcgtatcagagtcagacgttagcaagatcagagcagctaggcaggctcacttgaagtccttaggcttggaacaacccacaaactttaactatgctatttataaagcagtccagcccaccgctgggtgctaagtaactatataggcgaatgagagaaatatttgc

SEQ ID NO: 12 - RNA, 1470 nt, Tilapia lake virus
  source 1..1470: mol_type = viral cRNA
ccaaattttactctctattaccaaatacatttacttctgaaaatgagtcaatttgagaaatcattcaagggcagaactgaggtcacaataaccgaatatcgctctcatactgtcaaagatgtgcacagaagcttacttacggctgacaagtctctaaggaagtcattctgttttaggaacgccctaaaccagttcttggataaagatttgcctcttttgcccattcggccaaaattagagtccagggttgctgtgaaaaagtctaagctgaggagtcagctgtcgttcagacccggtttgactcaggaggaagcaattgatctttacaacaagggctatgatggtgacagcgtctcaggtgccttgcaagacagggtagtcaatgagcctgtagcttactcgagtgcagataatgacaaatttcacaggggcttagcggctctagggtacactttggctgatagagcatttgatacatgcgaatccggcttcgtgagagcaattcctaccactccatgcgggttcatatgttgtgggccaggttctttcaaagattcacttggatttgtgataaaaatcggcgaattctggcacatgtatgacgggttccaacacttcgtcgctgtcgaggatgctaagttcctagcaagtaagtctccttcgttttggttggcaaaacgtcttgcaaagaggctgaatctggtcccaaaagaggatccatctatagcagcagctgagtgcccttgtaggaaagtgtgggaagctagttttgctagggcacctactgcactagatccatttggaggcagggccttctgcgaccagggttgggtgtaccacagggacgtagggtatgcaacagctaaccacatatcacaggaaacactttttcaacaagcgctttcagtgaggaaccttggaccgcaaggtagtgcaaatgtctcaggctcaatacataccgccctggacaggctcagagcagcgtacagtagggggacgcccgcctctagatctatactgcaagggcttgcaaatctcatcacacctgtaggtgaaaactttgaatgcgatctcgacaagaggaagctcaatataaaggcattacgttctcccgagaggtacattacgatagagggcctggttgtaaacctggacgatgtggttagagggttctaccttgacaaggcgaaggtcactgttctctcgagatcaaagtggatgggttacgaggaccttcctcagaaacctccgaacggtacattttactgtagaaagaggaaggcaatgcttctcatctcatgtagtccaggcacgtacgcaaagaagcgaaaagtggcagtgcaggaggatcgctttaaagatatgagggttgagaatttccgggaggtagcggaaaatatggatctaaatcagtagggtttcttggcaaaagccttcactatatatatggtaataatgagaaagatttgc

SEQ ID NO: 13 - RNA, 1371 nt, Tilapia lake virus
  source 1..1371: mol_type = viral cRNA
ccaaatattaccccttaatccttaatagaccgttaactttcttttgaaatggactcgcggtttgcacagctaactggggttttctgtgacgatttcacttatagcgaagggagccgaaggttcctaagttcttacagtacagtagagagacgtccaggagtccccgtagagggtgactgttatgactgtttgaagaataagtggattgcctttgagctggaaggccagccgcggaaatttccaaaggcaacagttcgttgcattttgaacaatgatgctacatacgtttgctctgagcaagagtaccagcagatttgtaaggtacaattcaaggattatttggagatcgacggggttgttaaagttgggcacaaggcatcctacgatgctgagctaagggaacggctattggaactaccacatccaaagagtggcccgaagcctcgtattgagtgggtggcaccacccagacttgcggacatatccaaggaaacagctgagctaaagaggcaatatggattcttcgagtgctcaaagttcctcgcctgcggtgaggagtgtggtcttgaccaagaggcaagagaacttatactgaacgagtacgcacgtgatagagaatttgagttccgcaatggagggtggatacaaaggtatacagttgcttctcacaagcctgctacacagaagatattacctctaccggctagtgctccacttgctcgtgagcttttgatgttgattgctagaagcacaactcaggcagggaaagtactgcatagcgataataccagcatactagctgtaccggtcatgcgcgactctggaaagcacagtaaaaggagaccaaccgcctccactcaccacttagttgtaggtctaagtaaacctggctgtgaacacgattttgagtttgacgggtacagggcagctgtgcatgtgatgcacctagatcccaagcaatcggctaatataggggagcaagactttgtgagtacccgagaaatttacaagctggatatgttggaactacctcccataagtaggaagggtgatctggacagagctagtggtcttgagacaagatgggacgtcatcttacttctggaatgcctcgactctacaagggttagccaagcagtggctcaacattttaataggcaccggctagcacttagcgtctgtaaggacgagttcaggaaaggctaccagctggcttctgagataaggggtacaatacccttaagctcactttattattcactttgtgcagtaagattgcggatgacagtacacccatttgcgagatgatcgctttcgacgccttcgctaaaggttacgacgttctaatagaggattatgggaaaaatttgc

SEQ ID NO: 14 - RNA, 1250 nt, Tilapia lake virus
  source 1..1250: mol_type = viral cRNA
ccaaagtttactcctattacccagaatagctaacttaataaactgaaaatggtgagaactacaaagactagtatggcagctgccagcactgttgcaccagaggtagcaatggatgaaagttcacccagcacttcgcaggtacaagctgaactcccaagaaaccttgaggttttcaacgaagcttgtggtcatgtgtttggaagttcctttaacagggaggacaacagtgtgatatctgatgctgctgcatttctctttaaaatgcacactcactccctcgatggtcaggaggctaaggttctgagagccagtgaaaagaagagagagagggagaacgctaagaaatcaaggaaggcaccagaagcagggatgagggtcggaaggagccttatattaaccagcagatggactgaatactgcgcaacctgtgtgcctgcactgggctcaaagatgaaggtgataaaagcctcaggggacgcagctatgattcagatgatgaaggaccataactctctattaagagtgtgtgttcgcattgaggtctggaaggctaggtacgtcagtttggttgctctcgacgagaggattcagactttggaggacgcccaatggttcccatatctgagtggggattcctatcgtgcttgcccagggctggttggtggctactttgcaaagaaagcagcagcaggagaaagaggaaagaactacaaaaagttgaatcagactgctataatcccgcctccgagatttctgatcattggccacaggctgcagataggcgaccaggtcaccctcagggagctgcttgcctcaattgcttggggcctttgcgacggtgtccttgctgagtgttggagcccctcgcagggggacgggagtattggtgttgttgytggtctacctctgcaagctacaggaagctgcttcctggtggtagctagccacgggctctcagcaattgccgactcyaggattgagggaacagggaacacgaatcygctggaagaatgcattgccattcagaaacaggacggtgtcataaaatgtaagagaagtgggaagagtctgtatcactgcctcaaggagacagcaggggctgttgggagataggcaacgaagtaggccacccatgcgcggaaagctgcacaggctgccaagggcccctcttagcccaagttttctatatattctttaacaagtcatctaaaactggtaaattcttagacggtaattggagaaagatttgc

SEQ ID NO: 15 - RNA, 1098 nt, Tilapia lake virus
  source 1..1098: mol_type = viral cRNA
ccaaatgtttctcttagctcagactccaatagctatgcaggcgctggtcctgacaagctgcctagtttgcgcactagcaagtgatgaaagtttaaggataaaacgactacaatcatacctaaacaatacctaccaaagtagggagatagaaagtgaaataaggcgtggatttgcatccaagttcaggatggagagttgctcctgcactatgggggtgcactacattgtaaccccatcctcgggtgggtcgttctgcactgggttacatgcagtacctaacagcttcccagccctcgggtacaaacttcccaaagcaggggggagaggtgattggaaagctactgaagttaggattgacgaagatagtggggttgttctatacaacgtttccaggtgcagccacagtagcgagtgcagagatttggaggtgtattccaccgtactgccaggtcagtgtgactgtaccagacccactgtggacgactacaagaccatgctggcctcaaggcagccgaagtcgtttgtagtagcaggcctcattatactgtgtttacttgctagctcagtagcaattggcatgggtgtttacaattatgctggggtcatcggcctagcggacgcagctcaagcagatgtttctgagatttgggagtacttagaagctttgacacgggaagtcaccggtatgacgctaggagagttttgctcgattaaatccctcgtctgtaaatctgataacataggcaaattcaaagagcaatttgcagcctttggggaagctattcttgcaatagtgtttggratgctagagaaatataagtttgtctattacctggtgctttcgctgatggttctctcgctactcagtaaacttgttcctctgttgaagcaggtgcccttctatgggagtatcaaagttttagtattccggaggctaagagttgtgtgtttcaagacctttttctatattaagaagcggcttaagaagaaaagcccgcttgaggatgacgaagtccctctgcttccattatcttgatccttaagcttctattcctggtaggtcataaagtggtaaactgagaaaagagtaaaatttgc

SEQ ID NO: 16 - RNA, 1044 nt, Tilapia lake virus
  source 1..1044: mol_type = viral cRNA
ccaaattttacctctcgcatgcatttttatctacaggattgtccaatgagttggcttagggtgataagaacccttactttgttttcaacactctttaacggaagcgatcaatgcgtggacaacatgtggcggttttacgggagatcaaactacacgtcaagcgtagtcattgacggggacaaatattctgttgaaggttcatattcgagtagtgattatctggatccagcagtacagaaggtggttctgggacttgatggtagcaacgaagtcatagactcaggtgggtctccatactacatgtatgatttggagggatcaaaaggggaactccatcatctgaactgcaactttgtcgagaaacgatgtaatccgacgctaaactttatgcttggaggatttgttttgtgcccaggaatatcgagaaaagaactggagcctgtaaccgacaagatattggagagccggggaataccgggccgaggtaaaatacgtactataaaaataagctctaaactgtttgagacatcgctgtgcctttcgaagaggagrcccatctttagcacctgtatgctaatgtcgcgtggtctttgtacaaactgtaagcgtactatagatagaacatatatgacgccaaacggcttcagaactgaatacaagtggagctgcagagacaatagcacaaaacagtgttgggtattagttgagtcactggaggagaatcactccccatacaaatgccacttttccgcagtggaagtcctactaccagccgaaataaagcgtcaccagctcatcagcgagtggtccgcgatgcaggatgaagttgcttataagaagtcaaatgcttatcttcttgctcgtacttttcttagctatacaaaaatgcgtagattaaatcctgtaattgatctttcgatatcaccaccagtgacagtaagatcctgctgtaaaattaataaatacatgtgacactatatgtcctatcatgtgggcgaaatgcccaggtacagttttaagtgcttgattgagagaaatatttgc

SEQ ID NO: 17 - RNA, 777 nt, Tilapia lake virus
  source 1..777: mol_type = viral cRNA
ccaaattttactctctttgcattgcataccgtatagtaatagacacaatgtcctacaagattggtgagcttgagagaattatcacgcgcaaaaacaccctccccaaggacagcggaagtcagactgggctgttccatcgactgctcctagagcattactctggtgcctcgaacgtatggttcttttgtgcaactgggtttacacccaatacaaatggcacaacctggattgtattgacgagtcacccaaccgatggtggagaaaaggtacctttgaaatggaagtatgaagtgagccccggattgccagtcagaagggtacttgcccaggagggtacagcagtaagaggcccgaaaggagcctatttagtcaaaggggacatgcatctctgttcaactaccttctacactagaagrgaagcgaagtactggctctgtgcgccatccccaaagtttccacattggaccaagagatcagcgttggtgaccagcactcgaccactgactgagttgagcagggttgccacatacctagaggctataagtaagggtgcaactgatgtcaatgaatcgtggtgttcctaccacagagttgggttagtgccaatccctaaaggaatcacgtttgaactctaattacccagctgtttcgttgtcttattggggaggcctttctaagttaactaattacttttgaaagcagggataattggtctgagagcttcttatggtactcagtaccgttcactaaggatggtagcatgagagaaagatttgc

SEQ ID NO: 18 - RNA, 657 nt, Tilapia lake virus
  source 1..657: mol_type = viral cRNA
ccaaatattacctcatctacactaacatttccaattggacagcatatccaggaataagtatggctcaaatcccaacactaagagagggccaagggaagctctacgatttcacgctcaacggcatgacagtgactagagacacagtcaacactgtagttgctctggagtttcttgtcaatgcaggtccggatttgctttccctaacaattggcgaaggcctctcagaagaaacaaagtttaaacacctgcttgttaagcacgccggcatgacccgaaagcggatagaggaaaggctgggacgaatctcgaggcgagtcagtgtgacagtcgacgcaattataataacaaaccgcaagggtcaaagatttgaattcaatcggaagcagtacctggatattgccaaacaagctatgaagcttaagctccctgggattaactgtgtcgacatacccactgcgctcgcttttctcgaggaggtcctggcrrctgctttgaaggacactgaaggttcacaagatgacaggatggcccttaaggcagacacttctgctgctatcaatcatttccgtgaaatgcttaaataaaaagtgagtcttcagtgtcattttccccagggaggtaagctactccatttgtgtaatgatgagaaaaatttgc

SEQ ID NO: 19 - RNA, 548 nt, Tilapia lake virus
  source 1..548: mol_type = viral cRNA
ccaaattttactcacaagtccgattactttttccgcttggtgatgtcrcgatggatagaaaatacagattctgtgtcagtaatcttgacagagatgagtcggtcgtacgtcactttgtgccattaccccccttggagcttgtgctgcggcggcaagacatcacaacctggtcaagtctggatcctggatcgaaaacattgtctagaatgttcagagatctcagagttaatgacactgagtcagccaacttggcaggagagtgcaatggtgatagggagctgggtccaagtagtaacggagcacggaattttgcacacttcaacatcggaaaggcaggcaccaagaagggtcatgtggaggatatctgacatggctggcgatagaactttatgagggcggtccaggggcaattgaagctccgcgaacctactgattcctcagctagaacattgtagtgaaccatgtgacattaataatttagctttagtaaaaatggataagcttcagctctggcaaagtatgactttaaggacgtgagaaagatttgc

SEQ ID NO: 20 - RNA, 465 nt, Tilapia lake virus
  source 1..465: mol_type = viral cRNA
ccaaattttaaccctactaacaccaaatatagctataagccaggatgagtgtggcagattatttgtcaagtgacagtgactcgggggctgagagctcaggatgtttagtactaagaagtcggaagatcaagaagggcaagaaagctgcttcaaagaagcgaagttggaagaatgaaaggtatggtgctgacgagagcggtgaagatartatagagtggggtgacgaagtcgacctcgagatggacgactgtgattctgcaatcccagagtgggctagggttgatttcaatcccaaaaacagaagggacagagaggatgatgggcagagtgacctatctcgattttccgaagatttcggaaagaagtctcttgacgtgcagtcttagcaccttaatatcggttctgatttcgttcgtatccacaggccaacgctaactatacagggtgtcagagggaaagatttgc

SEQ ID NO: 21 - DNA, 22 bp, synthetic construct
  source 1..22: mol_type = other DNA; note = PCR primer
gaccacgcgtatcgatgtcgac

SEQ ID NO: 22 - DNA, 39 bp, synthetic construct
  source 1..39: mol_type = other DNA; note = PCR primer
gaccacgcgtatcgatgtcgacttttttttttttttttv

SEQ ID NO: 23 - DNA, 20 bp, synthetic construct
  source 1..20: mol_type = other DNA; note = PCR primer
gcaggaatgtgcctataggt

SEQ ID NO: 24 - DNA, 18 bp, synthetic construct
  source 1..18: mol_type = other DNA; note = PCR primer
gcattacgttctcccgag

SEQ ID NO: 25 - DNA, 22 bp, synthetic construct
  source 1..22: mol_type = other DNA; note = PCR primer
gctggatatgttggaactacct

SEQ ID NO: 26 - DNA, 18 bp, synthetic construct
  source 1..18: mol_type = other DNA; note = PCR primer
gcagataggcgaccaggt

SEQ ID NO: 27 - DNA, 23 bp, synthetic construct
  source 1..23: mol_type = other DNA; note = PCR primer
gactacaagaccatgctggcctc

SEQ ID NO: 28 - DNA, 22 bp, synthetic construct
  source 1..22: mol_type = other DNA; note = PCR primer
gatattggagagccggggaata

SEQ ID NO: 29 - DNA, 21 bp, synthetic construct
  source 1..21: mol_type = other DNA; note = PCR primer
gatggtggagaaaaggtacct

SEQ ID NO: 30 - DNA, 20 bp, synthetic construct
  source 1..20: mol_type = other DNA; note = PCR primer
gcgaaggcctctcagaagaa

SEQ ID NO: 31 - DNA, 25 bp, synthetic construct
  source 1..25: mol_type = other DNA; note = PCR primer
ggtgatgtcacgatggatagaaaat

SEQ ID NO: 32 - DNA, 22 bp, synthetic construct
  source 1..22: mol_type = other DNA; note = PCR primer
ggatgagtgtggcagattattt

SEQ ID NO: 33 - DNA, 20 bp, synthetic construct
  source 1..20: mol_type = other DNA; note = PCR primer
gatagggattctactgtcaa

SEQ ID NO: 34 - DNA, 22 bp, synthetic construct
  source 1..22: mol_type = other DNA; note = PCR primer
gcacctgagacgctgtcaccat

SEQ ID NO: 35 - DNA, 27 bp, synthetic construct
  source 1..27: mol_type = other DNA; note = PCR primer
cgtgcgtactcgttcagtataagttct

SEQ ID NO: 36 - DNA, 19 bp, synthetic construct
  source 1..19: mol_type = other DNA; note = PCR primer
acctagccttccagacctc

SEQ ID NO: 37 - DNA, 23 bp, synthetic construct
  source 1..23: mol_type = other DNA; note = PCR primer
gacttcccgtgtcaaagcttcta

SEQ ID NO: 38 - DNA, 23 bp, synthetic construct
  source 1..23: mol_type = other DNA; note = PCR primer
gctccacttgtattcagttctga

SEQ ID NO: 39 - DNA, 23 bp, synthetic construct
  source 1..23: mol_type = other DNA; note = PCR primer
gtaggaacaccacgattcattga

SEQ ID NO: 40 - DNA, 21 bp, synthetic construct
  source 1..21: mol_type = other DNA; note = PCR primer
gcatttcacggaaatgattga

SEQ ID NO: 41 - DNA, 24 bp, synthetic construct
  source 1..24: mol_type = other DNA; note = PCR primer
cgtccttaaagtcatactttgcca

SEQ ID NO: 42 - DNA, 22 bp, synthetic construct
  source 1..22: mol_type = other DNA; note = PCR primer
gatattaaggtgctaagactgc

SEQ ID NO: 43 - DNA, 24 bp, synthetic construct
  source 1..24: mol_type = other DNA; note = PCR primer
ccaaacgttatctcttaattacgc

SEQ ID NO: 44 - DNA, 36 bp, synthetic construct
  source 1..36: mol_type = other DNA; note = PCR primer
taatacgactcactatagcaaatatttctctcattc

SEQ ID NO: 45 - DNA, 28 bp, synthetic construct
  source 1..28: mol_type = other DNA; note = PCR primer
ccaaattttactctctattaccaaatac

SEQ ID NO: 46 - DNA, 20 bp, synthetic construct
  source 1..20: mol_type = other DNA; note = PCR primer
aggtcctcgtaacccatcca

SEQ ID NO: 47 - DNA, 28 bp, synthetic construct
  source 1..28: mol_type = other DNA; note = PCR primer
ccaaatattaccccttaatccttaatag

SEQ ID NO: 48 - DNA, 35 bp, synthetic construct
  source 1..35: mol_type = other DNA; note = PCR primer
taatacgactcactatagcaaatttttcccataat

SEQ ID NO: 49 - DNA, 24 bp, synthetic construct
  source 1..24: mol_type = other DNA; note = PCR primer
ccaaagtttactcctattacccag

SEQ ID NO: 50 - DNA, 35 bp, synthetic construct
  source 1..35: mol_type = other DNA; note = PCR primer
taatacgactcactatagcaaatctttctccaatt

SEQ ID NO: 51 - DNA, 24 bp, synthetic construct
  source 1..24: mol_type = other DNA; note = PCR primer
ccaaatgtttctcttatctcagac

SEQ ID NO: 52 - DNA, 35 bp, synthetic construct
  source 1..35: mol_type = other DNA; note = PCR primer
taatacgactcactatagcaaattttactcttttt

SEQ ID NO: 53 - DNA, 16 bp, synthetic construct
  source 1..16: mol_type = other DNA; note = PCR primer
ccaaatgtttctctta

SEQ ID NO: 54 - DNA, 17 bp, synthetic construct
  source 1..17: mol_type = other DNA; note = PCR primer
gcaaattttactctttt

SEQ ID NO: 55 - DNA, 21 bp, synthetic construct
  source 1..21: mol_type = other DNA; note = PCR primer
ccaaattttacctctcgcatg

SEQ ID NO: 56 - DNA, 36 bp, synthetic construct
  source 1..36: mol_type = other DNA; note = PCR primer
taatacgactcactatagcaaatatttctctcaatc

SEQ ID NO: 57 - DNA, 19 bp, synthetic construct
  source 1..19: mol_type = other DNA; note = PCR primer
ccaaattttacctctcgca

SEQ ID NO: 58 - DNA, 22 bp, synthetic construct
  source 1..22: mol_type = other DNA; note = PCR primer
gcaaatatttctctcaatcaag

SEQ ID NO: 59 - DNA, 24 bp, synthetic construct
  source 1..24: mol_type = other DNA; note = PCR primer
ccaaattttactctctttgcattg

SEQ ID NO: 60 - DNA, 35 bp, synthetic construct
  source 1..35: mol_type = other DNA; note = PCR primer
taatacgactcactatagcaaatctttctctcatg

SEQ ID NO: 61 - DNA, 26 bp, synthetic construct
  source 1..26: mol_type = other DNA; note = PCR primer
ccaaatattacctcatctacactaac

SEQ ID NO: 62 - DNA, 36 bp, synthetic construct
  source 1..36: mol_type = other DNA; note = PCR primer
taatacgactcactatagcaaatttttctcatcatt

SEQ ID NO: 63 - DNA, 24 bp, synthetic construct
  source 1..24: mol_type = other DNA; note = PCR primer
ccaaattttactcacaagtccgat

SEQ ID NO: 64 - DNA, 35 bp, synthetic construct
  source 1..35: mol_type = other DNA; note = PCR primer
taatacgactcactatagcaaatctttctcacgtc

SEQ ID NO: 65 - DNA, 26 bp, synthetic construct
  source 1..26: mol_type = other DNA; note = PCR primer
ccaaattttaaccctactaacaccaa

SEQ ID NO: 66 - DNA, 34 bp, synthetic construct
  source 1..34: mol_type = other DNA; note = PCR primer
taatacgactcactatagcaaatctttccctctg

SEQ ID NO: 67 - DNA, 6877 bp, synthetic construct
  source 1..6877: mol_type = other DNA; note = plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcagttaaccggagtactggtcgacctccgaagttgggggggccaaatgtttctcttagctcagactccaatagctatgcaggcgctggtcctgacaagctgcctagtttgcgcactagcaagtgatgaaagtttaaggataaaacgactacaatcatacctaaacaatacctaccaaagtagggagatagaaagtgaaataaggcgtggatttgcatccaagttcaggatggagagttgctcctgcactatgggggtgcactacattgtaaccccatcctcgggtgggtcgttctgcactgggttacatgcagtacctaacagcttcccagccctcgggtacaaacttcccaaagcaggggggagaggtgattggaaagctactgaagttaggattgacgaagatagtggggttgttctatacaacgtttccaggtgcagccacagtagcgagtgcagagatttggaggtgtattccaccgtactgccaggtcagtgtgactgtaccagacccactgtggacgactacaagaccatgctggcctcaaggcagccgaagtcgtttgtagtagcaggcctcattatactgtgtttacttgctagctcagtagcaattggcatgggtgtttacaattatgctggggtcatcggcctagcggacgcagctcaagcagatgtttctgagatttgggagtacttagaagctttgacacgggaagtcaccggtatgacgctaggagagttttgctcgattaaatccctcgtctgtaaatctgataacataggcaaattcaaagagcaatttgcagcctttggggaagctattcttgcaatagtgtttggaatgctagagaaatataagtttgtctattacctggtgctttcgctgatggttctctcgctactcagtaaacttgttcctctgttgaagcaggtgcccttctatgggagtatcaaagttttagtattccggaggctaagagttgtgtgtttcaagacctttttctatattaagaagcggcttaagaagaaaagcccgcttgaggatgacgaagtccctctgcttccattatctggcagcggcgcgaccaactttagcctgctgaaacaggcgggcgatgtggaagaaaacccgggcccggtgagcggctggcggctgttcaagaagattagctgacctttttctatattaagaagcggcttaagaagaaaagcccgcttgaggatgacgaagtccctctgcttccattatcttgatccttaagcttctattcctggtaggtcataaagtggtaaactgagaaaagagtaaaatttgcaataacccggcggcccaaaatgccgactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2025/0263674 A1
(43) Pub. Date: Aug. 21, 2025

(54) PLASMID-BASED REVERSE GENETICS SYSTEM FOR TILAPIA LAKE VIRUS (TiLV)

(71) Applicant: City University of Hong Kong, Hong Kong (CN)

(72) Inventors: Xingxing WANG, Hong Kong (CN); Nikolaus OSTERRIEDER, Hong Kong (CN)

(21) Appl. No.: 18/442,171

(22) Filed: Feb. 15, 2024

(51) Int. Cl. C12N 7/00; C12N 5/071; C12N 5/077

(52) U.S. Cl. CPC C12N 7/00; C12N 5/0656; C12N 5/0686; C12N 2760/00021; C12N 2760/00051

(57) ABSTRACT

The present disclosures relate to the establishment of a plasmid-based reverse genetic system for TiLV and use of the system to rescue recombinant and reporter TiLVs. The present disclosures also relate to the plasmids used for the rescue of TiLV and the recombinant and reporter TiLVs generated using the methods disclosed herein.

### REFERENCE TO SEQUENCE LISTING

The Sequence Listing identified as Sequence_Listing_P25681US00.xml; Size: 93 kilobytes; and Date of Creation: Feb. 1, 2024, filed herewith, is hereby incorporated by reference in its entirety.

### TECHNICAL FIELD

The present disclosures relate to the establishment of a reverse genetics system (RGS) for tilapia lake virus (TiLV).

### BACKGROUND

Tilapia lake virus (TiLV), the etiological agent of TiLV disease, is an emerging virus first identified in Israel in 2014. It is a single-stranded, negative-sense orthomyxo-like RNA virus with ten segments, which has been assigned taxonomically to the new family Amnoonviridae, genus Tilapinevirus, species Tilapinevirus tilapiae (ICTV). Since the first description of the virus and the disease, cases have been reported in Asia, Africa, and the Americas. Recently, China, the largest producer of tilapia, reported its first natural TiLV infection. It should also be noted that tilapia is one of the most important protein sources, especially in low-to middle-income countries. TiLV can cause up to 90% mortality in tilapia, and there is no cure for TiLV infection and no commercially available vaccine. Thus, TiLV poses a severe threat to the global tilapia industry and food security.

Currently, scientific knowledge and research relating to TiLV is very limited and many important knowledge gaps need to be filled. The molecular mechanisms underlying the replication and pathogenesis of this virus remain, for the most part, unknown. Reverse genetics (RG), an approach used to generate virus entirely from cloned cDNA, is arguably the most powerful tool for studying viruses and for vaccine development. Until now, there has been no RGS for TiLV, which has hampered efforts to understand its pathogenesis and to generate novel vaccines or antiviral therapeutics.

There are several challenges in establishing a RGS for TiLV: (i) TiLV is a fish RNA virus, and recovery of infectious virus in fish-derived cells that are permissive for TiLV is difficult, primarily because systems previously employed for related viruses make use of a bidirectional transcription system, which would require knowledge of the fish RNA polymerase I promoter. However, there is currently no information available on the polymerase I promoter of cells that allow TiLV replication; (ii) The functions of most TiLV genes and proteins are unknown, which presents a significant obstacle in developing an RGS; and (iii) The TiLV genome consists of ten segments, and the large number of segments adds to the difficulty in developing of an efficient procedure to generate virus from cloned cDNA.

There is a necessity for the establishment of a RGS and use thereof for recovering TiLV.

### SUMMARY

The present disclosure relates to the establishment of an RGS that allows the rescue of recombinant TiLV despite the fact that nature and functions of TiLV proteins are unknown. This is the first RGS for TiLV. Furthermore, the present disclosure relates to reporter-tagged TiLVs, which can be used to monitor virus infection both in vitro and in vivo.

In a first aspect, provided herein is a method for rescuing recombinant tilapia lake virus (TiLV), comprising:
i) preparing a plurality of expression plasmids for expressing the ten genomic segments of TiLV, wherein each of the plurality of expression plasmids comprises at least one cDNA sequence of the ten genomic segments of TiLV;
ii) introducing the plurality of expression plasmids into co-cultured Vero cells and fish-derived cells that are susceptible to TiLV infection;
iii) culturing the cells obtained in step ii) at a temperature around 28° C., thereby producing the recombinant TiLV; and optionally
iv) recovering the reassortant TiLV.

In certain embodiments, the plurality of expression plasmids are bi-directional expression plasmids which express all viral genomic RNAs (gRNAs) and viral mRNAs of TiLV.

In certain embodiments, the cDNA sequences of the ten genomic segments express all viral genomic RNAs (gRNAs) and viral mRNAs of TiLV.

In certain embodiments, the cDNA sequences of the ten genomic segments of TiLV have at least 90% identity to SEQ ID NOs:1-10, respectively.

In certain embodiments, the cDNA sequences of the ten genomic segments of TiLV have at least 90% identity to SEQ ID NOs:11-20, respectively.

In certain embodiments, the cDNA sequences of the ten genomic segments of TiLV comprise or consist of SEQ ID NO:1-10, respectively.

In certain embodiments, the cDNA sequences of the ten genomic segments of TiLV comprise or consist of SEQ ID NO:11-20, respectively.

In certain embodiments, the fish-derived cells are E11 cells derived from Channa striatus.

In certain embodiments, the co-cultured Vero cells and fish-derived cells are Vero E6 cells and E11 cells.

In a second aspect, provided herein is a method for producing a reporter-tagged TiLV, comprising:
i) preparing an expression plasmid comprising cDNA sequence of one genomic segment of TiLV and a reporter tag sequence that is integrated following a certain number of 3′ terminal nucleotides of the open reading frame (ORF) of the segment; and preparing a plurality of expression plasmids comprises cDNA sequences of at least one of the remaining nine genomic segments of TiLV;
ii) introducing all the expression plasmids prepared in step i) into co-cultured Vero cells and fish-derived cells that are susceptible to TiLV infection;
iii) culturing the cells obtained in step ii) at a temperature around 28° C., thereby producing the reporter-tagged TiLV.

In certain embodiments, the expression plasmids are bi-directional expression plasmids which express all viral genomic RNAs (gRNAs) and viral mRNAs of TiLV.

In certain embodiments, the cDNA sequences of the total ten genomic segments have at least 90% identity to SEQ ID NOs:1-10, respectively.

In certain embodiments, the cDNA sequences of the total ten genomic segments have at least 90% identity to SEQ ID NOs:11-20, respectively.

In certain embodiments, the cDNA sequences of the total ten genomic segments comprise or consist of SEQ ID NO:1-10, respectively.

In certain embodiments, the cDNA sequences of the total ten genomic segments comprise or consist of SEQ ID NO:11-20, respectively.

In certain embodiments, the fish-derived cells are E11 cells derived from Channa striatus.

In certain embodiments, the co-cultured Vero cells and fish-derived cells are Vero E6 cells and E11 cells.

In certain embodiments, the reporter tag sequence is HiBIT, GFP, NanoLuc, or mCherry.

In a third aspect, provided herein are recombinant, reassortant and reporter TiL Vs that are produced by the method according to any of the embodiments described herein.

In a fourth aspect, provided herein are vaccines, which comprise attenuated or inactivated recombinant and reporter TiLVs described herein, any one or more of the expression plasmids described herein, and the viral proteins expressed by any one or more of the expression plasmids described herein.

### BRIEF DESCRIPTION OF THE DRAWINGS

The above and other objects and features of the present disclosure will become apparent from the following description of the disclosure, when taken in conjunction with the accompanying drawings.

FIG. 1. Schematic diagram of the reverse genetics system for the rescue of TiLV. (A) The backbone of the designed plasmid is based on the pcDNA3.1 vector and the transcription units comprising the CMV promoter, the mouse RNA polymerase I terminator, a viral cDNA of TiLV in negative-sense orientation, the human RNA polymerase I promoter, and the BGH polyadenylation signal. (B) Ten plasmids designed for the generation of viral gRNAs and viral mRNAs are transfected into co-cultured Vero E6 cells and E11 cells. Subsequently, recombinant or reporter viruses are recovered.

FIG. 2. Schematic diagram of TiLV genome (specifically referencing the TiLV-Israel-HK strain). The genome features include a unique sequence of 5 nucleotides at the 3′ terminal (uni-5), a main open reading frame (ORF) and a unique sequence of 6 nucleotides at the 5′ terminal (uni-6). The polyadenylation signal sequences within the genomic segments of TiLV are indicated.

FIG. 3. Rescue of a HiBiT-tagged reporter TiLV. (A) Schematic of the HP5-HiBiT fusions encoding the polyprotein containing HP5, HiBiT and the self-cleaving 2A peptide where indicated. The HiBiT sequence was integrated following either the 50th or 100th amino acid of HP5, represented as aa50 or aa100 respectively. The terminal 80 nucleotides of the HP5 ORF locate upstream of 2A and downstream of HiBiT. (B) RT-PCR were performed to amplify the full-length segment 5 of the reporter viruses. The PCR products were separated on 1% agarose gel. M=marker (NEB 1 kb Plus DNA Ladder), T1=recombinant TiLV-1, T (S5)=reassortant TiLV-1 (S5), P3=3rd passage of reporter virus, P4=4th passage of reporter virus and P5=5th passage of reporter virus.

FIG. 4. Immunofluorescence assay for the detection of HiBiT-tagged viral protein in E11 cells. E11 cells were either mock-infected or infected with recombinant TiLV-1 (Til-4-2011), recombinant TiLV-2 (TiLV-Israel-HK), or reporter TiLV. After 2 days, cells were fixed and the HiBiT-tagged viral protein was identified using a mouse anti-HiBiT antibody (as the primary antibody) and the Alexa Fluor 488 goat anti-mouse IgG antibody (as the secondary antibody). The arrows point to some representative fluorescence staining in E11 cells.

### DETAILED DESCRIPTION

### Definitions

The use of the singular herein includes the plural (and vice versa) unless specifically stated otherwise.

As used herein, the term “about” or “around” refers to a ±10%, ±7%, ±5%, ±3%, ±1%, or ±0% variation from the nominal value unless otherwise indicated or inferred.

The term “recovering/rescuing a virus” used herein encompasses any process well known from the man skilled in the art allowing the generation of a viral clone from the genome of the virus.

“Percent identity” in the context of two or more nucleic acids or polypeptide sequences, refers to the percentage of nucleotides or amino acids that two or more sequences or subsequences contain which are the same. A specified percentage of nucleotides can be referred to such as: 90%, 91%, 92%, 93%, 94%, 95%, 96%, 97%, 98%, or 99% or more identity.

A “mutation” as used herein, refers to a change in nucleic acid relative to a reference sequence (which is preferably a naturally-occurring normal or “wild-type” sequence), and includes translocation, deletion, insertion, and substitution.

The method disclosed herein produces TiLV through reverse genetics techniques. In these techniques, the viruses are produced in culture hosts using a plurality of expression plasmids which comprise cDNA sequences of TiLV genomic segments.

In a first aspect, provided herein is a method for rescuing recombinant tilapia lake virus (TiLV), comprising:
i) preparing a plurality of expression plasmids for expressing the ten genomic segments of TiLV, wherein each of the plurality of expression plasmids comprises at least one cDNA sequence of the ten genomic segments of TiLV;
ii) introducing the plurality of expression plasmids into co-cultured Vero cells and fish-derived cells that are susceptible to TiLV infection;
iii) culturing the cells obtained in step ii) at a temperature around 28° C., thereby producing the recombinant TiLV; and optionally
iv) recovering the reassortant TiLV.

The following provides detailed description of the method.

### The Expression Plasmids

The plurality of expression plasmids express all the viral gRNAs and viral mRNAs for the generation of recombinant TiLV.

The expression plasmids are bi-directional expression plasmid.

In particular, a bi-directional expression plasmid contains at least two promoters which drive expression in different directions (i.e., both 5′ to 3′ and 3′ to 5′) from the same plasmid. Typically, one of the promoters is a pol I promoter and at least one of the other promoters is a pol II promoter. This is useful as the pol I promoter can be used to express uncapped viral RNAs while the pol II promoter can be used to transcribe mRNAs which can subsequently be translated into viral proteins, thus allowing simultaneous expression of RNA and protein from the same plasmid.

The pol I and pol II promoters used in the expression plasmids may be endogenous to an organism from the same taxonomic order from which the host cell is derived. Alternatively, the promoters can be derived from an organism in a different taxonomic order than the host cell.

The expression plasmids can be produced by synthesizing the cDNAs of each of the ten genomic fragments of TiLV. Then the cDNAs may be incorporated into a plasmid or other episomal construct, using conventional techniques known in the art.

The expression plasmids should encode all the genomic segments of TiLV which are necessary to produce a TiLV. Alternatively, the expression plasmid may encode one, two, three, four, five or more genomic segments.

In certain embodiments, the cDNA sequences of the ten genomic segments of TiLV have at least 90%, e.g., 91%, 92%, 93%, 94%, 95%, 96%, 97%, 98%, 99% or 100% identity to SEQ ID NOs:1-10 or SEQ ID NO: 11-20, respectively. Typically, any mutation in the cDNA sequence does not cause termination of amino acid translation. The plurality of expression plasmids should express all viral gRNAs and viral mRNAs of TiLV in order to produce the recombinant TiLV.

In certain embodiments, the cDNA sequences of the ten genomic segments of TiLV comprise or consist of SEQ ID NO:1-10 or SEQ ID NO:11-20, respectively.

### Transfection of Cells

The expression plasmids can be introduced into host cells using any technique known to those of skill in the art. For example, expression plasmids can be introduced into host cells by employing electroporation, DEAE-dextran, calcium phosphate precipitation, cationic lipid, or viral vectors. In certain embodiments, lipid-based transfection reagents such as lipofectamine 3000 are preferred. Once transfected, the host cells will begin to express the encoded viral RNA segments.

In certain embodiments, the cells for expressing the ten genomic segments of TiLV are Vero cells that are co-cultured with fish-derived cells that are susceptible to TiLV infection. For example, the Vero cells comprise Vero JCRB0111, Vero CCL-81, Vero 76 or Vero E6 cells.

In certain embodiments, the fish-derived cells that are susceptible to TiLV infection include E11, SSN-1, TiB (Tilapia brain), FHM (Fathead minnow), CIK (Ctenopharyngodon idella kidney), CCB (Common carp brain), and ZF4 (Zebrafish fry).

Where cells are used as a culture host, it is known that cell culture conditions (e.g., temperature, cell density, pH value, etc.) are variable over a wide range subject to the cell lines and can be adapted to the requirements of the application.

In certain embodiments, before transfection of the expression plasmids, the co-cultured cells are cultured at around 32-37° C., e.g., 33-37° C., 34-37° C., 35-37° C., 36-37° C., 32-36° C., 32-35° C., 32-34° C., or 32-33° C. Typically, the cells are cultured at about 37° C.

In certain embodiments, the cells are transfected with a total amount of 5 μg to 15 μg plasmids, with each plasmid accounting for 0.5-1.5 μg. In certain embodiments, total amount of all the plasmids for transfecting the cells is from 6 μg to 15 μg, 7 μg to 15 μg, 8 μg to 15 μg, 9 μg to 15 μg, 10 μg to 15 μg, 11 μg to 15 μg, 12 μg to 15 μg, 13 μg to 15 μg, 14 μg to 15 μg, etc.

In certain embodiments, after transfection of the plasmids, the cells are cultured at around 28° C., e.g., 30° C., 29° C., 27° C., 26° C., 25° C. or even lower temperature.

### Virus Harvest

In certain embodiments, the methods also include harvesting and isolating the recombinant viruses by known methods in the art.

In certain embodiments, the recovered recombinant TiLV viruses can be passaged on fish-derived cells that are susceptible to TiLV infection, such as E11, SSN-1 and TiB.

Reporter viruses are powerful tools for monitoring virus infection in both cultured cells and their animal hosts. It has proven particularly useful for screening antiviral agents in vitro or assessing efficacy of vaccines or therapeutics in vivo. Therefore, in a second aspect, provided herein is a method for producing a reporter-tagged TiLV, comprising:
i) preparing an expression plasmid comprising cDNA sequence of one genomic segment of TiLV and a reporter tag sequence that is integrated following a certain number of 3′ terminal nucleotides of the open reading frame (ORF) of the segment; and preparing a plurality of expression plasmids comprises cDNA sequences of at least one of the remaining nine genomic segments of TiLV;
ii) introducing all the expression plasmids prepared in step i) into co-cultured Vero cells and fish-derived cells that are susceptible to TiLV infection;
iii) culturing the cells obtained in step ii) at a temperature around 28° C., thereby producing the reporter-tagged TiLV.

In certain embodiments, an additional self-cleaving peptide such as 2A peptide is inserted between the ORF and the tag.

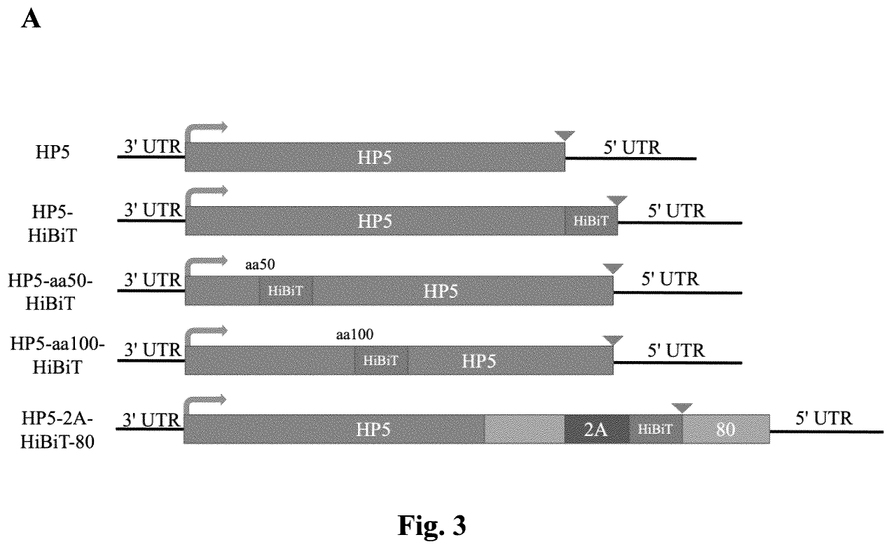

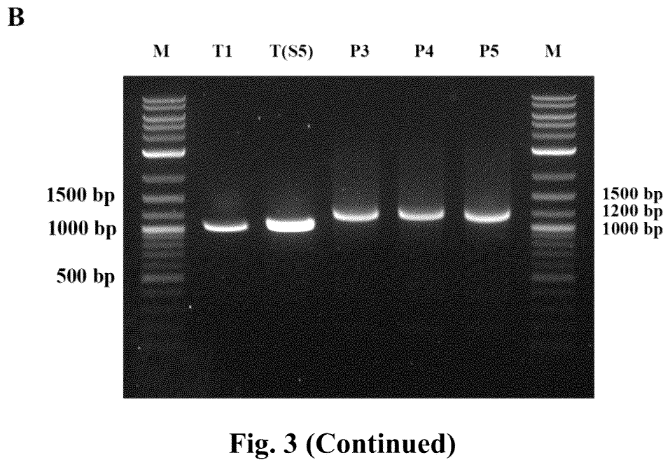

An example of integration of one tag into one genomic segment can refer to HP5-2A-HiBIT-80 in FIG. 3A, wherein 80 nucleotides at 3′ terminal of ORF of segment 5 are added to the 3′ end of the HiBiT tag. It should be understood that the nucleotides to be added to the 3′ end of the tag are associated with the packaging signal sequence of the segment and should encompass the packaging signal sequence specific to this segment.

In certain embodiments, the expression plasmids are bi-directional expression plasmids which express all viral gRNAs and viral mRNAs of TiLV.

In certain embodiments, the cDNA sequences of the total ten genomic segments have at least 90% identity to SEQ ID NOs:1-10, respectively.

In certain embodiments, the cDNA sequences of the total ten genomic segments have at least 90% identity to SEQ ID NOs:11-20, respectively.

In certain embodiments, the cDNA sequences of the total ten genomic segments comprise or consist of SEQ ID NO:1-10, respectively.

In certain embodiments, the cDNA sequences of the total ten genomic segments comprise or consist of SEQ ID NO:11-20, respectively.

In certain embodiments, the fish-derived cells are E11 cells derived from Channa striatus.

In certain embodiments, the co-cultured Vero cells and fish-derived cells are Vero E6 cells and E11 cells.

In certain embodiments, the reporter tag is HiBIT, GFP, NanoLuc, or mCherry. Advantageously, the reporter tag is a HiBiT tag. The reporter tags can be detected by known methods in the art.

In a third aspect, provided herein are recombinant, reassortant and reporter TiLVs which are generated by any methods disclosed herein.

The recombinant and reporter viruses are valuable tools for studying TiLV replication, pathogenesis, and host-virus interactions. They can be used to investigate the function of specific viral genes or to study the effects of viral infections on cells and organisms.

In a fourth aspect, provided herein are vaccines, which comprise attenuated or inactivated recombinant and reporter TiLVs described herein, any one or more of the expression plasmids described herein, and the viral proteins expressed by any one or more of the expression plasmids described herein.

### EXAMPLES

### Materials and Methods

### Cells and Viruses

Vero E6 cells (ATCC, CRL-1586) and E11 cells (derived from striped snakehead fish, generously provided by Dr. Sven M. Bergmann) were cultured in MEMα medium (Gibco) supplemented with 10% fetal bovine serum (FBS, Gibco) at the indicated temperatures under a 5% CO₂ atmosphere. The TiLV isolate (kindly provided by Prof. Eran Bacharach) was propagated in E11 cells, and the complete genome was determined as described below (this sequenced TiLV strain was named as TiLV-Israel-HK).

The ten genomic segments of tilapia lake virus isolate Til-4-2011 are shown in SEQ ID NOs: 1-10, and the ten genomic segments of tilapia lake virus isolate TiLV-Israel-HK are shown in SEQ ID NO: 11-20, respectively.

The primers for amplifying the terminals of mRNA or complementary RNA, the terminals of genomic RNA, and the full-length genomic segment of TiLV are listed in Table 1.

TABLE 1
Primer list

| Primer name | Primer sequence (5′ to 3′) | Purpose |
|---|---|---|
| PCR anchor primer | GACCACGCGTATCGATGTCGAC | |
| | (SEQ ID NO: 21) | |
| oligo(dT)-anchor | GACCACGCGTATCGATGTCGACTT | V = A, C, or G |
| primer | TTTTTTTTTTTTTTV | |
| | (SEQ ID NO: 22) | |
| S1-m&cRNA | GCAGGAATGTGCCTATAGGT | Primers used to |
| | (SEQ ID NO: 23) | amplify the |
| S2-m&cRNA | GCATTACGTTCTCCCGAG | terminals of |
| | (SEQ ID NO: 24) | mRNA or |
| S3-m&cRNA | GCTGGATATGTTGGAACTACCT | complementary |
| | (SEQ ID NO: 25) | RNA of TiLV. |
| S4-m&cRNA | GCAGATAGGCGACCAGGT | |
| | (SEQ ID NO: 26) | |
| S5-m&cRNA | GACTACAAGACCATGCTGGCCTC | |
| | (SEQ ID NO: 27) | |
| S6-m&cRNA | GATATTGGAGAGCCGGGGAATA | |
| | (SEQ ID NO: 28) | |
| S7-m&cRNA | GATGGTGGAGAAAAGGTACCT | |
| | (SEQ ID NO: 29) | |
| S8-m&cRNA | GCGAAGGCCTCTCAGAAGAA | |
| | (SEQ ID NO: 30) | |
| S9-m&cRNA | GGTGATGTCACGATGGATAGAAAAT | |
| | (SEQ ID NO: 31) | |
| S10-m&cRNA | GGATGAGTGTGGCAGATTATTT | |
| | (SEQ ID NO: 32) | |
| S1-gRNA | GATAGGGATTCTACTGTCAA | Primers used to |
| | (SEQ ID NO: 33) | amplify the |
| S2-gRNA | GCACCTGAGACGCTGTCACCAT | terminals of |
| | (SEQ ID NO: 34) | genomic RNA of |
| S3-gRNA | CGTGCGTACTCGTTCAGTATAAGTTCT | TILV. |
| | (SEQ ID NO: 35) | |
| S4-gRNA | ACCTAGCCTTCCAGACCTC | |
| | (SEQ ID NO: 36) | |
| S5-gRNA | GACTTCCCGTGTCAAAGCTTCTA | |
| | (SEQ ID NO: 37) | |
| S6-gRNA | GCTCCACTTGTATTCAGTTCTGA | |
| | (SEQ ID NO: 38) | |
| S7-gRNA | GTAGGAACACCACGATTCATTGA | |
| | (SEQ ID NO: 39) | |
| S8-gRNA | GCATTTCACGGAAATGATTGA | |
| | (SEQ ID NO: 40) | |
| S9-gRNA | CGTCCTTAAAGTCATACTTTGCCA | |
| | (SEQ ID NO: 41) | |
| S10-gRNA | GATATTAAGGTGCTAAGACTGC | |
| | (SEQ ID NO: 42) | |
| S1-FL-F | CCAAACGTTATCTCTTAATTACGC | Primer pairs used |
| | (SEQ ID NO: 43) | to amplify the full- |
| S1-FL-R | TAATACGACTCACTATAGCAAATATTTCTCTCATTC | length genomic |
| | (SEQ ID NO: 44) | segment of TiLV |
| S2-FL-F | CCAAATTTTACTCTCTATTACCAAATAC | with the exception |
| | (SEQ ID NO: 45) | of segment 2. |
| S2-Part-R | AGGTCCTCGTAACCCATCCA | |
| | (SEQ ID NO: 46) | |
| S3-FL-F | CCAAATATTACCCCTTAATCCTTAATAG | |
| | (SEQ ID NO: 47) | |
| S3-FL-R | TAATACGACTCACTATAGCAAATTTTTCCCATAAT | |
| | (SEQ ID NO: 48) | |
| S4-FL-F | CCAAAGTTTACTCCTATTACCCAG | |
| | (SEQ ID NO: 49) | |
| S4-FL-R | TAATACGACTCACTATAGCAAATCTTTCTCCAATT | |
| | (SEQ ID NO: 50) | |
| S5-FL-F | CCAAATGTTTCTCTTATCTCAGAC | |
| | (SEQ ID NO: 51) | |
| S5-FL-R | TAATACGACTCACTATAGCAAATTTTACTCTTTTT | |
| | (SEQ ID NO: 52) | |
| S5-FL-F-2 | CCAAATGTTTCTCTTA | |
| | (SEQ ID NO: 53) | |
| S5-FL-R-2 | GCAAATTTTACTCTTTT | |
| | (SEQ ID NO: 54) | |
| S6-FL-F | CCAAATTTTACCTCTCGCATG | |
| | (SEQ ID NO: 55) | |
| S6-FL-R | TAATACGACTCACTATAGCAAATATTTCTCTCAATC | |
| | (SEQ ID NO: 56) | |
| S6-FL-F-2 | CCAAATTTTACCTCTCGCA | |
| | (SEQ ID NO: 57) | |
| S6-FL-R-2 | GCAAATATTTCTCTCAATCAAG | |
| | (SEQ ID NO: 58) | |
| S7-FL-F | CCAAATTTTACTCTCTTTGCATTG | |
| | (SEQ ID NO: 59) | |
| S7-FL-R | TAATACGACTCACTATAGCAAATCTTTCTCTCATG | |
| | (SEQ ID NO: 60) | |
| S8-FL-F | CCAAATATTACCTCATCTACACTAAC | |
| | (SEQ ID NO: 61) | |
| S8-FL-R | TAATACGACTCACTATAGCAAATTTTTCTCATCATT | |
| | (SEQ ID NO: 62) | |
| S9-FL-F | CCAAATTTTACTCACAAGTCCGAT | |
| | (SEQ ID NO: 63) | |
| S9-FL-R | TAATACGACTCACTATAGCAAATCTTTCTCACGTC | |
| | (SEQ ID NO: 64) | |
| S10-FL-F | CCAAATTTTAACCCTACTAACACCAA | |
| | (SEQ ID NO: 65) | |
| S10-FL-R | TAATACGACTCACTATAGCAAATCTTTCCCTCTG | |
| | (SEQ ID NO: 66) | |

### Plasmids

The cDNA of each TiLV segment (SEQ ID NO: 1-10) was synthesized and introduced into pcDNA3.1 vector at sites flanked by the 222 bp of human RNA polymerase I (Pol 1) promoter sequence and the 34 bp of murine terminator sequence. The resulting plasmids were synthesized by BGI and were named pPolI-TiLV-S1, pPolI-TiLV-S2, pPolI-TiLV-S3, pPolI-TiLV-S4, pPolI-TiLV-S5, pPolI-TiLV-S6, pPolI-TiLV-S7, pPolI-TiLV-S8, pPolI-TiLV-S9, and pPolI-TiLV-S10.The synthetic sequences of TiLV segments were identical to the TiLV reference genome (NCBI accession no: GCF_001630085.1), with G chosen for R (G or A) at nucleotide position 481, A for R at nucleotide position 661, G for R at nucleotide position 703 of segment 3.

Three plasmids were constructed based on our TiLV genome sequencing results. The synthetic sequences of TiLV segments 2, segment 5, and segment 6 in plasmids pPolI-TiLV-S2, pPolI-TiLV-S5, and pPolI-TiLV-S6 were replaced with new sequences (SEQ ID NO:12, SEQ ID NO: 15 and SEQ ID NO: 16) from the TiLV-Israel-HK strain using overlap extension PCR cloning. The resulting plasmids were named pPolI-TiLV-S2-2, pPolI-TiLV-S5-2, and pPolI-TiLV-S6-2.

The plasmids used for generating reporter virus were synthesized by BGI and was named pPolI-HP5-2A-HiBiT80 (SEQ ID NO: 67). All plasmids used for transfection were isolated by the Plasmid Maxi or Midi Kit (QIAGEN).

### Determination of the Complete Viral Genome

The 5′ and 3′ terminal sequences of TiLV were determined by 3′ RACE using the 5′/3′ RACE Kit (2^{nd} Generation, Roche). Firstly, total RNA was extracted from TiLV-infected E11 cells using TRIzol Reagent (Invitrogen) and polyadenylated using Poly (A) Polymerase (NEB). The polyadenylated RNA was used for first-strand cDNA synthesis with oligo (dT)-anchor primer (See Table 1). The synthesized cDNA was then used as the template to amplify the terminal sequences with the PCR anchor primer and gene-specific primers (GSPs) (See Table 1). All target PCR products were purified by the QIAquick Gel Extraction Kit (QIAGEN) and were subjected to Sanger sequencing.

According to the sequencing results of 3′ RACE, primers (See Table 1) were designed to amplify the full-length of TiLV segments. Similarly, target PCR products were purified and were subjected to Sanger sequencing.

### 3′ RACE for Determining the Polyadenylation Signal Sequence

The 3′ terminal sequences of TiLV mRNA were determined by 3′ RACE using the total RNA isolated from E11 cells infected by the wild-type TiLV (TiLV-Israel-HK). Unlike the above procedure, the total RNA was not polyadenylated. All other steps are almost the same as above described.

Recovery of Recombinant, Reassortant and Reporter TiLVs From Cloned cDNAs

Vero E6 cells and E11 cells (at a ratio of approximately 4:1) were seeded in T25 flasks and cultured at 37° C. overnight. The co-cultured cells were transfected with 10 plasmids (each 1.25 μg) using 2 μL of Lipofectamine 3000 Transfection Reagent (Invitrogen) per microgram of plasmid DNA. The transfected cells were then incubated at 28° C.

### Sanger Sequencing of Segments 5 and 6 for Recombinant, Reassortant and Reporter TiLVs

Recovered viruses were passaged on E11 cells. At 3 days post-infection, total RNA was isolated from E11 cells infected by the recovered viruses (at least passage 3) using the TRIzol Reagent, and cDNA was synthesized using the High-Capacity cDNA Reverse Transcription Kit (Applied Biosystems). Touchdown PCR was performed to amplify the full-length of segments 5 and 6. Target PCR products were purified and were subjected to Sanger sequencing.

### Next-Generation Sequencing of a Recombinant TiLV

Total RNA was isolated from E11 cells infected with recombinant TiLV-1 (Til-4-2011). The library preparation, Illumina sequencing (pair-end sequencing of 151 bp) and basic de novo assembly were done at Centre for PanorOmic Sciences (CPOS) of the University of Hong Kong.

### Immunofluorescence (IF) Assay for Reporter TiLV Detection

The mouse anti-HiBiT monoclonal antibody (Promega) was used for the identification of reporter TiLV-infected cells. Cells were fixed with ice-cold methanol (Sigma) for 15 min at −20° C., then blocked with 1% bovine serum albumin (BSA, Sigma) containing 0.3% Triton X-100 (Sigma) for 1 h at room temperature. After that, the cells were incubated with the anti-HiBiT antibody at a dilution of 1:1000 in 1% BSA containing 0.3% Triton X-100 for 2 h at room temperature. After three consecutive washing steps with PBS, the cells were incubated with Alexa Fluor 488 goat anti-mouse IgG antibody (Invitrogen) at a dilution of 1:1 000 in 1% BSA containing 0.3% Triton X-100 for 1 h at room temperature. Finally, the cells were washed with PBS three times as above, and images were captured using a Nikon ECLIPSE Ti2-E inverted microscope.

### Results

### Development of a Reverse Genetics System for TiLV

In efforts to develop an RGS for TiLV, the susceptibility of many mammalian cell lines such as HEK293T, Calu-3, Vero E6, BHK21, and swine testicular (ST) to TiLV infection was tested. Ultimately, it is discovered that TiLV can replicate in Vero E6 cells. It is surmised, therefore, that TiLV can be rescued by transfecting Vero E6 cells. Due to the limited information available on the function of TiLV proteins, a bidirectional transcription system to generate influenza viruses was employed. The strategy involved introducing all viral genomic RNA (gRNA) segments and viral mRNAs into TiLV permissive cells using this approach.

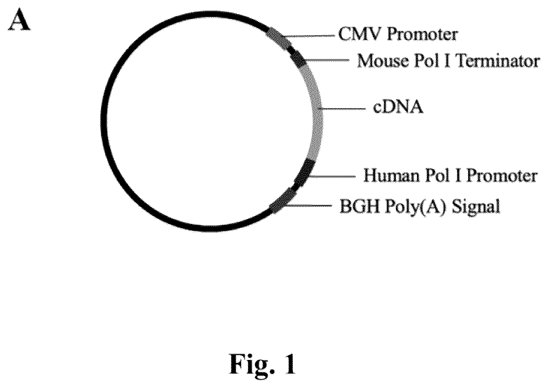

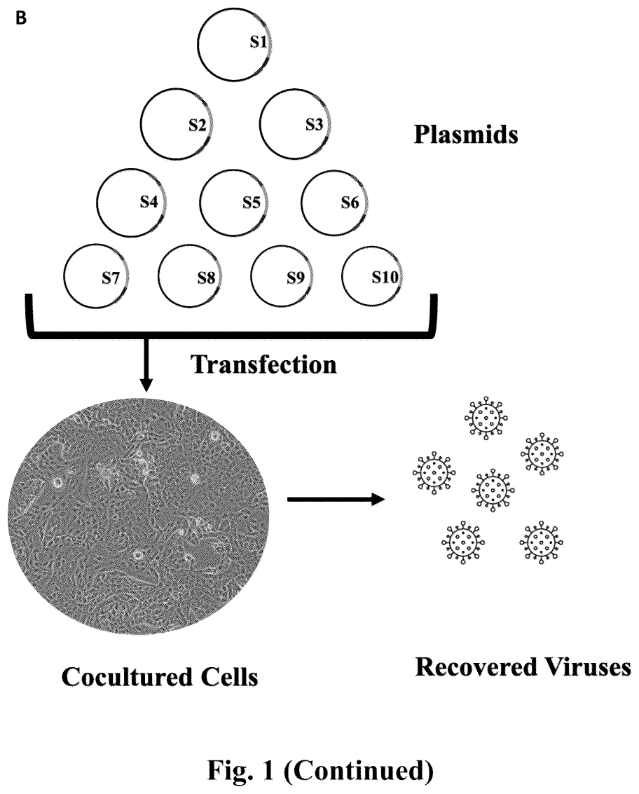

First, a plasmid was designed in which the cDNA of each TiLV segment was inserted between an RNA polymerase I promoter and an RNA polymerase II promoter (FIG. 1A). The insertion of the 10 TiLV cDNAs was expected to result in transcription of the 10 viral gRNAs and all viral mRNAs. Ten plasmids containing the cDNAs of the TiLV reference genome (GCF_001630085.1, TiLV isolate Til-4-2011) were synthesized and transfected into Vero E6 cells that were co-cultured with fish-derived E11 cells (FIG. 1B, Table 2). The transfected cells were cultured at 28° C., and supernatants were collected at different time points to test for the presence of recovered viruses in E11 cells. Starting from 7 days post-transfection, it was observed transfected cells released recovered viruses in numbers sufficient for detection in the cells. Recovered viruses were then passaged on E11 cells and two methods were used to verify that the recovered viruses indeed were from cloned cDNA and represented the designed virus (Til-4-2011): (i) The full-length cDNAs of segments 5 and 6 of the recovered virus were produced by RT-PCR and sequenced. Sequence analysis confirmed that the sequences for segments 5 and 6 were identical to the cDNAs of the plasmids used for generating this designed virus, and (ii) Next-generation sequencing was performed using total RNA isolated from E11 cells infected with this recovered virus (raw sequencing reads are available at NCBI and can be accessed with BioProject No. PRJNA1010714). De novo assembly was performed and of the 15 assembled scaffolds with the highest read coverages, ten were found to be highly similar (approximately 100%) to the 10 TiLV segments (Table 3). Taken together, these results show that the virus from cloned cDNA indeed corresponded to the designed virus (Til-4-2011).

TABLE 2
Plasmids used for generating recombinant, reassortant and reporter TiLVs

| recombinant | recombinant | reassortant | reassortant | |
|---|---|---|---|---|
| TiLV-1 | TiLV-2 | TiLV-1 | TiLV-2 | |
| (Til-4-2011) | (TiLV-Israel-HK) | (S5) | (S6) | reporter TiLV |
| pPolI-TiLV- | pPolI-TiLV-S1 | pPolI-TiLV-S1 | pPolI-TiLV-S1 | pPolI-TiLV-S1 |
| S1 | | | | |
| pPolI-TiLV- | pPolI-TiLV-S2-2 | pPolI-TiLV-S2 | pPolI-TiLV-S2 | pPolI-TiLV-S2-2 |
| S2 | | | | |
| pPolI-TiLV- | pPolI-TiLV-S3 | pPolI-TiLV-S3 | pPolI-TiLV-S3 | pPolI-TiLV-S3 |
| S3 | | | | |
| pPolI-TiLV- | pPolI-TiLV-S4 | pPolI-TiLV-S4 | pPolI-TiLV-S4 | pPolI-TiLV-S4 |
| S4 | | | | |
| pPolI-TiLV- | pPolI-TiLV-S5-2 | pPolI-TiLV- | pPolI-TiLV-S5 | pPolI-HP5-2A- |
| S5 | | S5-2 | | HiBiT80 |
| pPolI-TiLV- | pPolI-TiLV-S6-2 | pPolI-TiLV-S6 | pPolI-TiLV-S6-2 | pPolI-TiLV-S6-2 |
| S6 | | | | |
| pPolI-TiLV- | pPolI-TiLV-S7 | pPolI-TiLV-S7 | pPolI-TiLV-S7 | pPolI-TiLV-S7 |
| S7 | | | | |
| pPolI-TiLV- | pPolI-TiLV-S8 | pPolI-TiLV-S8 | pPolI-TiLV-S8 | pPolI-TiLV-S8 |
| S8 | | | | |
| pPolI-TiLV- | pPolI-TiLV-S9 | pPolI-TiLV-S9 | pPolI-TiLV-S9 | pPolI-TiLV-S9 |
| S9 | | | | |
| pPolI-TiLV- | pPolI-TiLV-S10 | pPolI-TiLV-S10 | pPolI-TiLV-S10 | pPolI-TiLV-S10 |
| S10 | | | | |

TABLE 3
Summary of the top 15 scaffolds from the de novo assembly

| | Scaffold | TiLV reference | Reads | | Percentage |
|---|---|---|---|---|---|
| | length | segment length | coverage for | BLAST result | identity |
| Scaffold | (bp) | (bp) | assembly* | (NCBI Accession Number) | from BLAST |
| scaffold_55556 | 598 | 548 | 1,069,961 | Tilapia lake virus isolate | 100% |
| | | | | Til-4-2011 segment 9 (NC_029925.1) | |
| scaffold_53218 | 618 | 465 | 1,048,445 | Tilapia lake virus isolate | 99.78% |
| | | | | Til-4-2011 segment 10 | |
| | | | | (NC_029930.1) | |
| scaffold_42292 | 733 | 657 | 848,522 | Tilapia lake virus isolate | 100% |
| | | | | Til-4-2011 segment 8 (NC_029929.1) | |
| scaffold_18957 | 1,254 | 1,044 | 712,218 | Tilapia lake virus isolate | 100% |
| | | | | Til-4-2011 segment 6 (NC_029928.1) | |
| scaffold_13319 | 1,542 | 1,371 | 509,164 | Tilapia lake virus isolate | 99.78% |
| | | | | Til-4-2011 segment 3 (NC_029927.1) | |
| scaffold_10695 | 1,745 | 1,641 | 487,765 | Tilapia lake virus isolate | 100% |
| | | | | Til-4-2011 segment 1 (NC_029926.1) | |
| scaffold_17261 | 1,326 | 1,250 | 353,432 | Tilapia lake virus isolate | 100% |
| | | | | Til-4-2011 segment 4 (NC_029922.1) | |
| scaffold_34455 | 853 | 777 | 305,468 | Tilapia lake virus isolate | 100% |
| | | | | Til-4-2011 segment 7 (NC_029924.1) | |
| scaffold_13226 | 1,548 | 1,471 | 254,358 | Tilapia lake virus isolate | 100% |
| | | | | Til-4-2011 segment 2 (NC_029921.1) | |
| scaffold_12082 | 1,631 | NA | 85,975 | Channa striata isolate (MN057623.1) | 99.88% |
| scaffold_262 | 6,388 | 1,099 | 75,816 | Tilapia lake virus isolate | 99.91% |
| | | | | Til-4-2011 segment 5 (NC_029923.1) | |
| scaffold_126 | 7,620 | NA | 43,674 | Brama japonica voucher SIO | 92.22% |
| | | | | (OP151200.1) | |
| scaffold_13258 | 1,546 | NA | 28,414 | Xanthia icteritia genome assembly, | 90.00% |
| | | | | chromosome: 23 (OX421953.1) | |
| scaffold_89093 | 4,22 | NA | 23,968 | Epinephelus fuscoguttatus DNA | 91.61% |
| | | | | (AP022677.1) | |
| scaffold_55040 | 6,02 | NA | 17,810 | Plectropomus leopardus neuroblast | 82.53% |
| | | | | differentiation-associated protein | |
| | | | | (XM_042483312.1) | |

*No. of paired reads mapped to assembly × 151/scaffold length

To further confirm the utility of our established system, we sequenced the complete genome of a wild-type TiLV strain originating from Israel (named TiLV-Israel-HK). Since we had successfully recovered TiLV using the developed system, we hypothesized that complementary RNA (cRNA) is produced during TiLV replication. By adding poly (A) tails to the gRNA and CRNA, 3′ rapid amplification of cDNA ends (3′ RACE) can be used to determine the 3′-terminal sequences of gRNA and cRNA. Thus, we were in a position to determine the 5′-and 3′-terminal sequences of the TiLV genome segments. Combined with amplifying the full-length cDNA of each TiLV segment, the complete TiLV genome of TiLV-Israel-HK was assembled (SEQ ID NO: 11-20; GenBank accession numbers: OQ437054-OQ437063). Our sequence analysis revealed that segments 2, 5 and 6 have many differences when compared to the published reference genome of TiLV, while the sequences of the other seven segments were almost identical to the TiLV reference genome. Therefore, we constructed three plasmids containing cDNA based on our newly sequenced TiLV genome for segments 2, 5 and 6. We then transfected the previously synthesized plasmids (representing segments 1, 3, 4 and 7 to 10) and the three new plasmids into co-cultured Vero E6 cells and E11 cells (Table 2). Recovered viruses were passaged in E11 cells and the full-length cDNAs of segments 5 and 6 were amplified and sequenced. Sequence analysis of the amplified segments demonstrated that the new recombinant TiLV (TiLV-Israel-HK) was also successfully recovered.

### Identification of the TiLV Polyadenylation Signal

The polyadenylation signal of influenza viruses is a stretch of uridines located near the 5′ terminal of the genomic RNA. However, the sequence analysis of the TiLV genome did not reveal a similar pattern. This discrepancy led to further investigate the polyadenylation signal in TiLV. Given that the viral mRNAs of influenza viruses possess a poly (A) tail, it was hypothesized that the viral mRNAs of TiLV might also exhibit this feature. Finally, the 3′ terminal sequences of 10

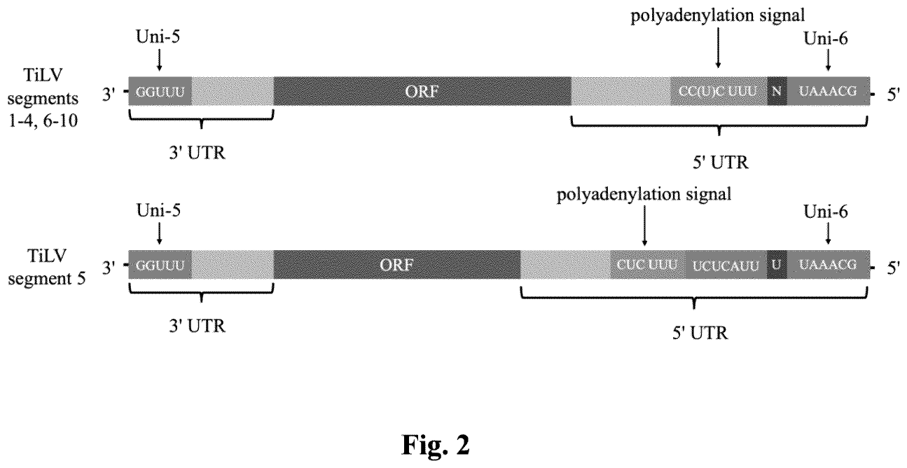

TiLV mRNAs were determined by 3′ RACE. The sequence analyses revealed that the polyadenylation signal sequence for the 10 TiLV segments is either 3′-CCC UUU-5′ or 3′-CUC UUU-5′. Segment 5 stands out with its unique location of the polyadenylation signal sequence, with 14 nucleotides following the polyadenylation signal. In contrast, the remaining 9 segments show more consistency, each having 7 nucleotides after the polyadenylation signal sequence (FIG. 2).

### Rescue of Reassortant TiLVs

Phylogenomic studies have provided evidence of reassortment in TiLV, particularly with regard to segments 5 and 6. To provide direct experimental proof for the generation of reassortant viruses using the developed RGS, nine plasmids containing the cDNA of Til-4-2011 were transfected with one plasmid containing the cDNA of either segment 5 or segment 6 of the TiLV-Israel-HK (Table 2). The recovery of reassortant viruses was confirmed by Sanger sequencing, which verified the presence of the TiLV-Israel-HK segments in the Til-4-2011 background. These results demonstrate that the established system allows for recovery of reassortant viruses and further confirms its utility and versatility.

### Engineering of a HiBiT-Tagged Reporter TiLV

Reporter viruses are powerful tools for monitoring virus infection in both cultured cells and their animal hosts. It has proven particularly useful for screening antiviral agents in vitro or assessing the efficacy of vaccines or therapeutics in vivo. However, the insertion of a reporter gene into viral genomes, especially in the case of (segmented) RNA viruses, can negatively affect critical biological features, making the development of a reliable reporter virus challenging. We, therefore, opted for the insertion of the HiBiT tag, an 11-amino-acid peptide sequence that enables easy detection and quantification of HiBiT-tagged proteins, to generate a reporter TiLV.

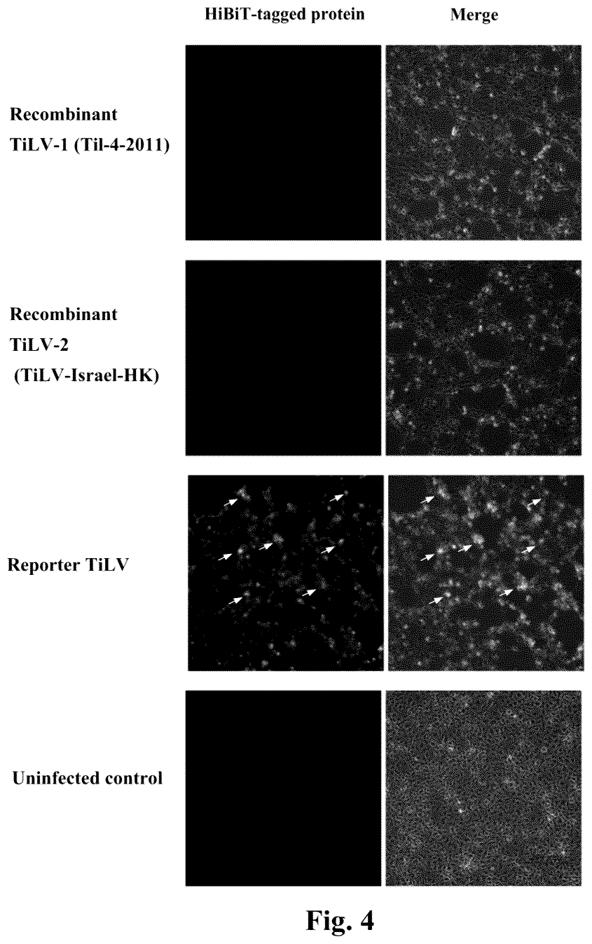

Initially, we attempted to add the HiBiT sequence directly to the predicted C-terminus of the open reading frame (ORF) encoded by TiLV segment 5 (FIG. 3A). However, despite several attempts, we were unable to generate a reporter virus. We then selected two insertion sites (FIG. 3A) that we predicted would have minimal impact on the putative function of the viral protein, but this approach also proved unsuccessful. Given that the genome of influenza viruses contains segment-specific packaging signal sequences that are essential for vRNA incorporation into virions, we hypothesized that similar packaging signals may exist in TiLV. To test this hypothesis, we designed a plasmid (pPolI-HP5-2A-HiBiT80) in which the terminal 80 nucleotides of the ORF of segment 5 were added after the HiBiT sequence to restore the packaging signal (FIG. 3A). Using this construct and the other nine unmodified plasmids, we could successfully generate a reporter virus. This reporter virus was passaged in E11 cells and caused obvious CPE that was similar to that observed with wild-type virus and the other rescued viruses. We employed RT-PCR to amplify the full-length segment 5 of the reporter-tagged viruses. As anticipated, gel electrophoresis imaging indicated that the size of the full-length segment 5 of the reporter TiLV is larger than that of the other unmodified rescued viruses (FIG. 3B). This validates the successful incorporation of our modifications into the TiLV genome. Furthermore, sequencing of segment 5 from the 3^{rd}, 4^{th}, and 5^{th} passage reporter viruses revealed that all sequences were identical to the cDNA of pPolI-HP5-2A-HiBiT80 and that the 80 nucleotides repeat sequence remained intact. We concluded from the results that the generated reporter virus is stable over serial passages. In addition, we performed immunofluorescence (IF) assay for the reporter virus. As shown in FIG. 4, the HiBiT-tagged viral protein was readily detected in E11 cells using an anti-HiBiT antibody.

In the experiments, we first tested the susceptibility of many mammalian cell lines to TiLV infection and discovered that TiLV can replicate in Vero E6 cells. Second, to address the unknown functions of TiLV genes and proteins, we introduced all viral gRNAs and viral mRNAs into Vero E6 cells. Third, to solve the problem caused by the large number of segments, we transfected Vero E6 with E11 cells, which present one of the most permissive cells for TiLV. We also increased the number of cells and the amounts of plasmids used for transfection to optimize yields.

The successful development of this TiLV RGS suggests that the replication mechanisms of TiLV are similar to those of influenza viruses. Our genome sequencing results indicate that viral cRNAs also exist during TiLV replication. However, the exact replication mechanisms require further investigation. Additionally, TiLV exhibits some unique features. For instance, we identified a distinct polyadenylation signal sequence in the TiLV genome through sequencing the terminal sequences of viral mRNAs. This signal sequence differs from those found in other RNA viruses.

The present invention is significant in that we have successfully established the plasmid-based RGS for TiLV. In summary, the developed RGS for TiLV will contribute to further research into the replication mechanisms of TiLV and enable thorough characterization of this virus. Significantly, it will pave the way for the creation of novel vaccines and antiviral treatments.

What is claimed is:

1. A method for rescuing a recombinant tilapia lake virus (TiLV), comprising:
i) preparing a plurality of expression plasmids for expressing ten genomic segments of TiLV, wherein each of the plurality of expression plasmids comprises at least one cDNA sequence of the ten genomic segments of TiLV;
ii) introducing the plurality of expression plasmids into co-cultured Vero cells and fish-derived cells that are susceptible to TiLV infection;
iii) culturing the cells obtained in step ii) at a temperature around 28° C., thereby producing the recombinant TiLV; and optionally
iv) recovering the reassortant TiLV.

2. The method of claim 1, wherein the plurality of expression plasmids are bi-directional expression plasmids which express all viral genomic RNAs (gRNAs) and viral mRNAs of TiLV.

3. The method of claim 1, wherein cDNA sequences of the ten genomic segments of TiLV have at least 90% identity to SEQ ID NOs:1-10, respectively.

4. The method of claim 1, wherein cDNA sequences of the ten genomic segments of TiLV have at least 90% identity to SEQ ID NOs:11-20, respectively.

5. The method of claim 1, wherein cDNA sequences of the ten genomic segments of TiLV comprise or consist of SEQ ID NO:1-10, respectively.

6. The method of claim 1, wherein cDNA sequences of the ten genomic segments of TiLV comprise or consist of SEQ ID NO:11-20, respectively.

7. The method of claim 1, wherein the fish-derived cells are E11 cells derived from Channa striatus.

8. The method of claim 1, wherein the co-cultured Vero cells and fish-derived cells are Vero E6 cells and E11 cells.

9. A method for producing a reporter-tagged TiLV, comprising:
i) preparing an expression plasmid comprising cDNA sequence of one genomic segment of TiLV and a reporter tag sequence that is integrated following a certain number of 3′ terminal nucleotides of the open reading frame (ORF) of the segment; and preparing a plurality of expression plasmids comprises cDNA sequences of at least one of the remaining nine genomic segments of TiLV;
ii) introducing all the expression plasmids prepared in step i) into co-cultured Vero cells and fish-derived cells that are susceptible to TiLV infection; and
iii) culturing the cells obtained in step ii) at a temperature around 28° C., thereby producing the reporter-tagged TiLV.

10. The method of claim 9, wherein the expression plasmids are bi-directional expression plasmids which express all viral genomic RNAs (gRNAs) and viral mRNAs of TiLV.

11. The method of claim 9, wherein cDNA sequences of the total ten genomic segments have at least 90% identity to SEQ ID NOs:1-10, respectively.

12. The method of claim 9, wherein cDNA sequences of the total ten genomic segments have at least 90% identity to SEQ ID NOs:11-20, respectively.

13. The method of claim 9, wherein cDNA sequences of the total ten genomic segments comprise or consist of SEQ ID NO:1-10, respectively.

14. The method of claim 9, wherein the cDNA sequences of the total ten genomic segments comprise or consist of SEQ ID NO:11-20, respectively.

15. The method of claim 9, wherein the fish-derived cells are E11 cells derived from Channa striatus.

16. The method of claim 9, wherein the co-cultured Vero cells and fish-derived cells are Vero E6 cells and E11 cells.

17. The method of claim 9, wherein the reporter tag sequence is HiBIT, GFP, NanoLuc, or mCherry.

18. A reporter TiLV that is produced by the method according to claim 9.
